(19) United States
(12) Patent Application Publication
(10) Pub. No.: US 2026/0232450 A1
(43) Pub. Date: Aug. 13, 2026

(54) Additive Manufactured Prosthesis

(71) Applicant: Addman Intermediate Holdings, LLC, Fort Myers, FL (US)

(72) Inventor: Mark Saberton, Bonita Springs, FL (US)

(21) Appl. No.: 19/532,528

(22) Filed: Feb. 6, 2026

Related U.S. Application Data
(60) Provisional application No. 63/755,331, filed on Feb. 7, 2025.

(51) Int. Cl. A61F 2/38; B33Y 10/00; B33Y 80/00; A61F 2/30

(52) U.S. Cl. CPC A61F 2/3859; B33Y 10/00; B33Y 80/00; A61F 2002/30112; A61F 2002/30985; A61F 2002/3863; A61F 2310/00161

(57) ABSTRACT

An additive manufactured knee femoral component replacement for either a full or partial replacement has an outer wall formed from a flexible polymer and a structural body or medial portion formed from a fiber reinforced polymer. The replacement can have a single posterior condyle or a pair of spaced apart condyles with a gap therebetween to accommodate ligaments extending therethrough.

### CROSS-REFERENCE TO RELATED APPLICATION

This application is a nonprovisional of, and claims priority to, co-pending U.S. Provisional Patent Application Serial No. 63/755,331, filed February 7, 2025, the entire disclosure of which is hereby incorporated by reference, in including the drawing.

### TECHNICAL FIELD

The present disclosure concerns joint replacement prostheses. More particularly, the present disclosure concerns implant materials for use in joint replacement surgeries. Even more particularly, the present disclosure concerns replacement of metal materials for use in joint replacement surgeries.

### BACKGROUND

As is known to those skilled in the art to which the present invention pertains, there has been an ongoing proliferation of joint replacement for knees and hips, as well as shoulders and the like.

Designing implant protheses for either a partial or total knee replacement, is done with the knowledge that, typically, the femoral condyles of the replacement are inserted into the femur and the femoral condyles mate with the patella and the meniscus, and relative movement of these structures occurs during flexing of the knee. Thus, the chosen prosthetic must address this issue by imparting little friction to the flexing movement. Yet, the prosthetic must also be durable. This combination of properties has yet to be achieved with known polymeric prosthetics, which are more desirable than metal prosthetics.

Quite often, due to arthritis or other conditions, partial or total replacement is necessary. Ordinarily, such replacements are done using metals such as titanium. Recently, however, much attention has been paid to the use of polymeric resins, such as PEEK (polyether ether ketone) or CFR-PEEK (carbon fiber reinforced polyether ether ketone) as an alternative to metals. The utilization of such a polymeric resin provides many advantages in that it can be additively manufactured or 3D printed and custom designed and printed and is easily manufactured. Furthermore, the replacement can be manufactured using multiple approaches including FFF or FDM and SLS, as well as SLA and Dynamic Light Processing (DLP).

The advantage to using polymers, such as PEEK and CFR-PEEK is that they are resistant to fatigue strain and they are radiologically transparent. They have mechanical properties and are suitable for a range of orthopedic applications. Further, they are potentially stress reducing shields, wear-resistant and preclude the release of metal ions by replacing the metal articulating components, while weighing less than the metal replacement.

Total or partial knee replacement generally involves the replacement of three components which cooperate together to form the joint, including: a) the femoral component with the condyles; b) a tibia insert with a bearing plate that replaces the meniscus and tibial cartilage; and c) the tibial component or base plate that replaces the tibia.

Typically, PEEK is preferred in such joint replacement procedures because of its non-abrading or lubricity when in contact with the meniscus, i.e., there is less friction. Oftentimes though, it can become necessary to increase the strength of such prosthetics which, depending on the environment, renders the CFR-PEEK type of polymer more desirable. Heretofore, the art has taught the use of one or the other, and there is a need for a polymer-based replacement implant with a superior combination of high strength, durability, and good friction properties.

### BRIEF SUMMARY

General aspects of the present disclosure relate to implant structures that incorporate both PEEK and CFR-PEEK for use in joint replacements, including knees, shoulders, etc., as well as methods of producing such structures, as detailed below.

Aspects of the disclosure relate to a femoral knee replacement implant that includes a curvilinear base plate having at least one curvilinear posterior condyle extending upwardly therefrom and a medial portion for encircling at least a portion of a femur and for underlying a patella. The implant has an outer surface formed from a flexible polymer and an infill portion formed from a fiber-reinforced polymer, and the flexible polymer has greater flexibility and lower friction properties relative to the fiber-reinforced polymer. The implant being additive manufactured.

According to one aspect, the implant includes a pair of spaced apart femoral condyles having a space therebetween to accommodate ligaments passing therethrough. In one configuration, the implant also includes at least one fiber-reinforced polymer upstanding post configured for connection to the femur. In this configuration, the implant may be a full knee replacement implant.

According to additional aspects, the implant is a partial knee replacement implant having a single posterior condyle or a single anterior condyle.

According to a further aspect, the flexible polymer is selected from the group consisting of PEEK, polycaprolactone, polylactic acid and polyglycolic acid, and the fiber-reinforced polymer includes a same material as the flexible polymer having carbon fiber reinforcement.

Additional aspects of the disclosure relate to a method for additive manufacturing a femoral component implant, which includes:
(a) creating an outer wall of a flexible polymer from a series of at least three layers of beads of a suitable material, the layers having alternating widths to create an interstitial gap between a first lowermost layer, a second layer above the first lowermost layer and a third layer above the second layer, the first layer and third layer each having a width greater than a width of the second layer to create a gap at termini of the first and third layers, such that the first and third layers are greater width layers, and the second layer is a lesser width layer;
(b) depositing a layer of strengthening infill into the gap between the greater width layers; and
(c) repeating steps (a) and (b) sequentially to create gaps between the alternating greater width layers at the termini; and thereafter depositing a layer of strengthening infill in the respective repeated gaps.

The strengthening infill extends into the gaps and into abutment with ends of the lesser width bead layers. The greater width layers have equal numbers of beads, and the lesser width layers have equal numbers of beads. Each bead in each layer is an oval bead, and a bead height of the beads in a lowermost layer alternate between a first height H1 and a second height H. In one aspect, each bead in each layer above the lowermost layer is of equal height, and may also be of equal width as well. In another aspect, the flexible polymer is PEEK and the infill is CFR-PEEK.

Further aspects of the disclosure relate to a skeletal replacement implant configured for connection to a bone, including a structural body formed of a first polymeric material, and an outer layer of a second polymeric material disposed on at least a portion of the structural body to form at least a portion of an outer surface of the implant. The structural body is configured to be fixedly connected to the bone to mount the implant on the bone. The at least a portion of the outer surface of the implant formed by the outer layer includes a bearing surface configured to moveably engage an adjacent skeletal structure. The first polymeric material has greater strength compared to the second polymeric material, and the second polymeric material provides a lower friction surface compared to the first polymeric material.

According to one aspect, the first polymer material and the second polymer material share a matrix material, and the first polymer material further includes fiber reinforcement. In one configuration, the matrix material is selected from the group consisting of PEEK, polycaprolactone, polylactic acid and polyglycolic acid. For example, the matrix material may be PEEK, such that the first polymer material is carbon fiber-reinforced PEEK, and the second polymer material is PEEK.

According to another aspect, the outer layer is disposed around an entire periphery of the structural body, such that the structural body forms an inner core of the implant.

According to a further aspect, the outer layer is disposed on less than an entirety of the outer periphery of the structural body, such that the outer layer is disposed at least on the bearing surface and is not disposed at least a portion of the structural body configured for connection to the bone.

According to yet another aspect, the implant is a femoral replacement implant having at least one condyle with an outwardly-curved outer surface at least partially defining the bearing surface, and the outer layer is disposed on the outwardly-curved outer surface of the at least one condyle. In one configuration, the implant has two condyles spaced by a gap, each condyle having an outwardly-curved outer surface at least partially defining the bearing surface, and the outer layer is disposed on both of the outwardly-curved outer surfaces of the two condyles.

According to a still further aspect, the structural body and the outer layer are formed together by an additive manufacturing technique.

Other features and advantages of the disclosure will be apparent from the following description taken in conjunction with the attached drawings.

### BRIEF DESCRIPTION OF THE DRAWINGS

FIG. 1 illustrates the skeletal posterior of a knee joint;

FIG. 2 is a perspective view of one embodiment of a CFR-PEEK femoral replacement implant depicting femoral condyles according to aspects of the present disclosure;

FIG. 3 is a sectional view of the femoral replacement implant of FIG. 2; and

FIG. 4 is an exploded cross-sectional view of a portion of the replacement implant shown in FIG. 3.

FIG. 5 is a cut-away perspective view of a leg of the femoral replacement implant thereof;

FIG. 6 is a partial cross-sectional view taken along line 6-6 of FIG. 2;

FIG. 7 is a perspective view of a partial anterior condyle replacement; and

FIG. 8 is a schematic top-front view of one embodiment of a femoral replacement implant according to aspects of the present disclosure.

### DETAILED DESCRIPTION

At the outset, it is noted that due to their inherent strength, CFR-PEEK implants can work very well with static or non-moving implants, such as cranial, facial or spine implants. However, the addition of carbon fiber in a PEEK moving implant, such as a knee, hip or shoulder, causes unnecessary friction in the surface-to-surface interface of moving parts. Contrariwise, unreinforced PEEK implants exhibit very low friction and are therefore superior as an implant for moving parts, i.e., joints, but lack the strength of fiber-reinforced PEEK implants.

As detailed hereinafter, the present disclosure, in a first aspect, describes a replacement implant having the low friction advantage of pure PEEK with the strength of a fiber-reinforced PEEK implant.

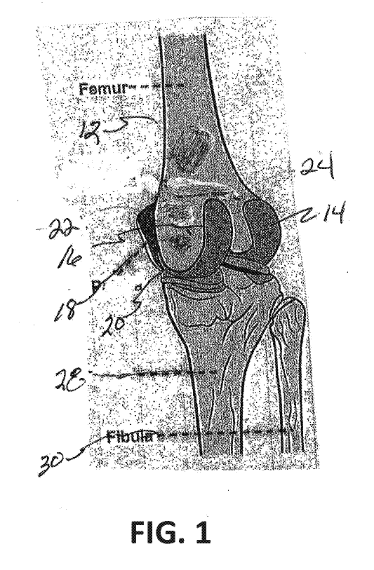

Now, and with reference to the drawings, and in particular, FIG. 1, there is depicted an illustration of a knee joint, generally denoted at 10. The joint 10 includes a femur 12 having a femoral component which includes posterior femoral condyles 14, 16. The anterior femoral component (not shown) can be a unitary component or may comprise spaced apart condyles depending upon the degree of wear of the femoral component.

A patella or kneecap 18 overlies the anterior portion of the femur 12. A medial portion 20 contacts the meniscus and lies thereatop. The tibia and fibula 28, 30 underlie the meniscus, as shown.

In accordance with the present disclosure and with reference to FIGS. 2 – 4, there is shown, a posterior femoral knee replacement appliance or implant, generally, denoted at 32.

The replacement implant 32 conforms to a typical configuration of a femur as shown in FIG. 1, having both a posterior and anterior curvilinear configuration 33. Typically, the posterior portion of a femur replacement has two spaced apart curvilinear condyles 34, 36 with a space 19 (intercondylar fossa) therebetween through which the cruciate ligaments extend. The replacement 32 includes a medial portion or section 38.

A pair of spaced apart posts 40, 42 extend upwardly from the medial section 38 of the replacement 32 and are insertable into a patient’s femur 12 by any suitable means, to secure the femoral component replacement 32 into position. Preferably, the posts 40, 42 are printed from CFR-PEEK. In other embodiments, the replacement 32 may be connected to the patient’s femur 12 by other means, which may or may not utilize integral posts 40, 42, e.g., various fasteners and other mechanical connectors, biocompatible adhesives or other bonding materials, etc. In such a configuration, the posts 40, 42 may be absent.

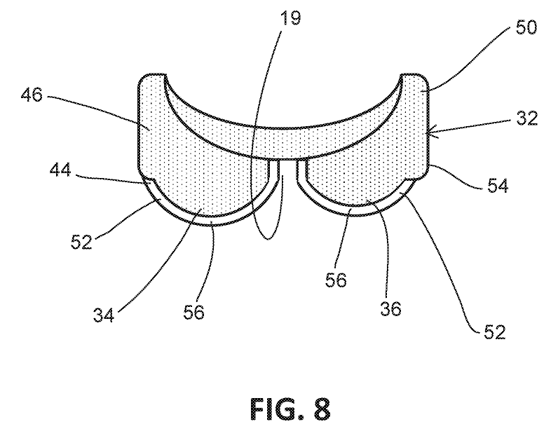

FIG. 8 illustrates one embodiment of a replacement 32 schematically, with the understanding that the structure of the replacement 32 in FIG. 8 may be adapted to a shape suitable for use as a full or partial knee replacement implant or a different type of skeletal implant. As shown schematically in FIG. 8, the replacement 32 may be constructed to have a structural body 50 formed of a first polymeric material with an outer layer 52 of a second polymeric material disposed on the structural body 50 to form at least a portion of an outer surface 54 of the replacement 32. In general, the structural body 50 may be formed of a material (e.g., CFR-PEEK) having superior physical properties, such as strength (i.e., tensile strength), hardness, and/or fracture toughness, compared to the outer layer 52, while the outer layer 52 may be formed of a material (e.g., PEEK) having lower friction and/or higher lubricity compared to the structural body 50. It is understood that, conversely, the material of the structural body 50 may create a higher friction surface than the material of the outer layer 52, while the material of the outer layer 52 may be more flexible. In one embodiment, the first and second polymeric materials may have the same polymer matrix, e.g., the first polymeric material may be a composite having a matrix material, while the second polymeric material may be the matrix material without reinforcement, a different composite sharing the same matrix material, or a mixture of polymer materials including the matrix material. An example configuration uses CFR-PEEK as the first polymeric material of the structural body 50 and PEEK as the second polymeric material of the outer layer 52.

In one embodiment (not shown), the outer layer 52 may be disposed around an entire periphery of the structural body 50, such that the structural body 50 forms an inner core of the replacement 32. In another embodiment, the outer layer 52 may be disposed on only a portion of the outer periphery of the structural body 50. For example, the outer layer 52 may be disposed on contact surfaces or bearing surfaces 56 of the implant 32 that are designed for moving contact with another skeletal structure such as bone, cartilage, etc., i.e., at a joint, such as shown in FIG. 8. In this configuration, some or all other portions of the structural body 50 may have no outer layer 52 disposed thereon. In a femoral replacement implant 32 such as shown in FIG. 8, the outer layer 52 may be disposed at least on the curved outer surfaces of the condyles 34, 36 and optionally on surfaces surrounding or adjacent to such contact surfaces. In this embodiment, at least some of the surfaces of the replacement 32 that fixedly and/or non-moveably engage the bone to which the replacement 32 is fixed may have no outer layer 52, such that the structural body 50 is exposed. Other surfaces that do not moveably engage another skeletal structure (e.g., a knee meniscus) may or may not have an outer layer 52 as desired. In one embodiment, surfaces of the replacement 32 that are designed to non-moveably engage the bone may have no outer layer 52, such that the structural body 50 is fixed directly to the bone. For example, in an implant 32 such as shown in FIG. 2, the posts 40, 42 and surrounding surfaces may have no outer layer 52 disposed thereon in one embodiment.

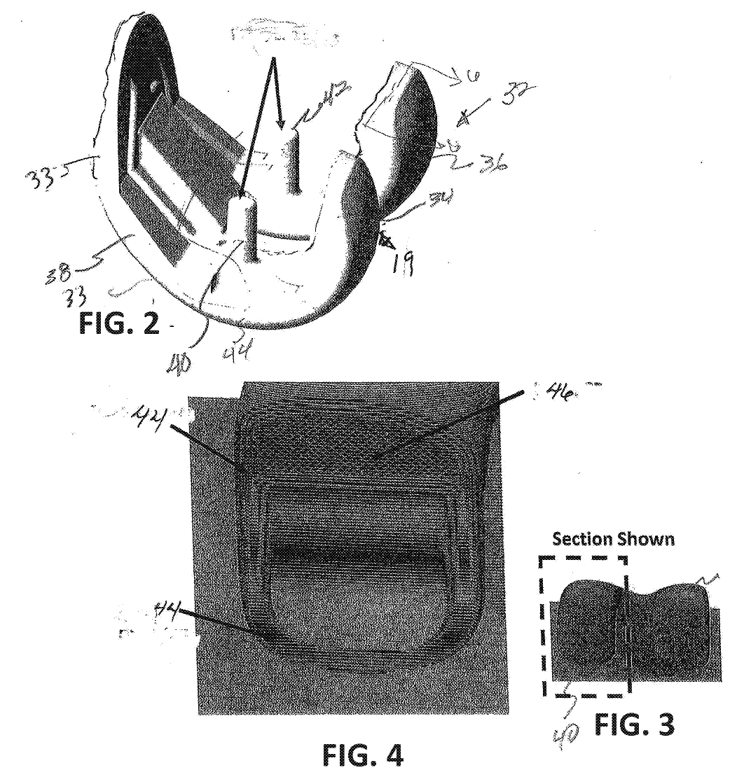

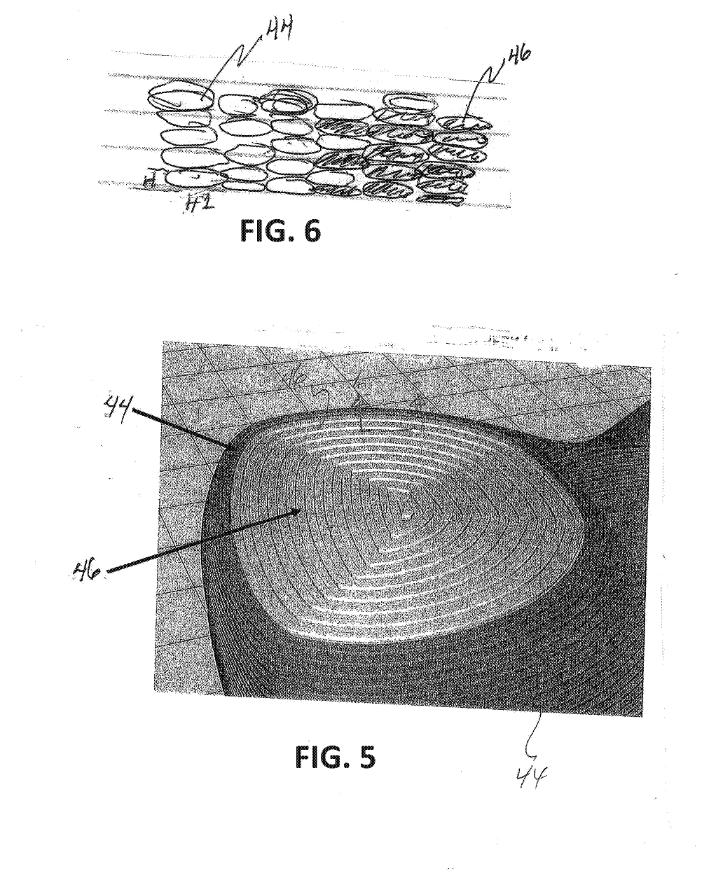

A structure having the structural body 50 and the outer layer 52 as disclosed herein may be manufactured using additive manufacturing by forming an outer wall 44 using the second polymer material and an interior or infill portion 46 using the first polymer material. As shown in FIGS. 3 - 5, the replacement 32, itself, has a peripheral outer wall 44 and an interior or infill portion 46. The replacement 32 is additively manufactured in accordance herewith, wherein the outer wall 44 is printed substantially contemporaneously layer-by-layer with the interior internal infill structure or portion 46.

In order to secure the additive manufactured PEEK outer wall 44 to the interior infill 46, an interlocking CFR-PEEK infill defining the interior 46 is used. The infill interlocking is such as that disclosed in U.S. Patent No. 12,017,407, the disclosure of which is hereby incorporated by reference and as shown in FIG. 6 and includes alternating bead heights H and H1. According to the methods disclosed in the ‘407 patent, the manufacturing method may include:
(a) creating an outer wall of a flexible polymer from a series of at least three layers of beads of a suitable material, the layers having alternating widths to create an interstitial gap between a first lowermost layer, a second layer above the first lowermost layer and a third layer above the second layer, the first layer and third layer each having a width greater than a width of the second layer to create a gap at termini of the first and third layers, such that the first and third layers are greater width layers, and the second layer is a lesser width layer;
(b) depositing a layer of strengthening infill into the gap between the greater width layers; and
(c) repeating steps (a) and (b) sequentially to create gaps between the alternating greater width layers at the termini; and thereafter depositing a layer of strengthening infill in the respective repeated gaps;
where the strengthening infill extends into the gaps and into abutment with ends of the lesser width bead layers, and further wherein the greater width layers have equal numbers of beads, and the lesser width layers have equal numbers of beads, each bead in each layer being an oval bead and further wherein a bead height of the beads in a lowermost layer alternate between a first height H1 and a second height H. In one embodiment, each bead in each layer above the lowermost layer is of equal height, and may be of both equal height and width.

The interlocking of the outer wall 44 of the polymer to the infill portion 46 is achieved by filling the gaps between the layers as taught in the ‘407 patent and as shown in FIG. 6, hereof.

The use of the same or similar matrix materials for the outer wall 44 and the infill portion 46 may enhance bonding therebetween.

By printing the replacement device in this manner, only the PEEK outer wall 44 engages the bearing surfaces of a meniscus. If replacement of the meniscus and a tibia bearing plate are required in the knee replacement procedure, the tibia replacement implant can be manufactured in accordance with the same additive manufacturing process as disclosed herein.

In manufacturing the femoral replacement 32, as noted, an additive manufacturing process is employed which generally comprises modeling the femoral joint to be replaced using an FDM (FFF), STL, SLA, SLS, DLP procedure or the like for modeling. The configuration for the femoral replacement can also be manually modeled.

Preferably, FDM is used for printing the present joint replacement. As is known, FDM printers extrude the PEEK and/or CFR-PEEK filament in a layer-by-layer extrusion over a a build plate according to the model.

The so-modeled replacement is then transmitted to a slicer. The slicer configures the model for printing using standard slicing techniques, such as tessellating, to configure the replacement for printing. The slicer then transmits the configured model to a printer which, in turn, per the instructions from the slicer, controls the jets or nozzle(s) for the layer-by-layer deposition.

Further, in manufacturing the present prosthetic, a conventional 3D printing process with a single nozzle printer or a multi-nozzle printer may be used.

Multi-nozzle fusion machines are well-known and commercially available and can be mated such that as the layers are being deposited atop one another, the deposition of the PEEK is contemporaneous with the deposition of the CFR-PEEK using separate jets.

In practicing the present invention, and in printing the present implant, it is understood that the medial femoral condyle (MFC) is wider and has a larger articulating surface area than the lateral condyle (LFC), although the LFC is larger anteroposteriorly. The anterior articular surfaces of the condyles become less convex and form a V-shaped groove, known as the trochlear sulcus, that articulates with the patella. Posteriorly, the condyles are separated by the intercondylar fossa, which becomes the intercondylar notch anteriorly, through which run the cruciate ligaments.

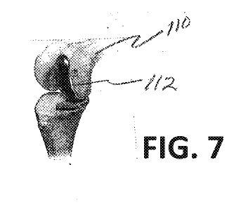

As noted above, replacements 32 according to the present disclosure may be a partial knee replacement or other joint replacement. Referring to FIG. 7, a flexed femur 110 has an anterior articulating replacement condyle implant 112 secured thereto in the manner heretofore described and which underlies a patella (not shown). The condyle implant 112 may be manufactured using techniques disclosed herein, and may have a structure as disclosed herein.

It should be noted that although the present invention has been described with respect to PEEK and CFR-PEEK, it is possible to use alternative printable materials which overcome the potential biodegradability problems with PEEK implants. Thus, it is contemplated that the present invention can be used with polymers other than PEEK, such as polylactic acid, polyglycolic acid and polycaprolactone since these polymers degrade safely within the body.

Other potentially useful biomaterials include, for example, poly2-metha-cryloxyethyl phosphorylcholine, polycarbonate urethane, ultra-high molecular weight polyethylene, and

polymethacrylate, as well as other semi-crystalline polymers. Other potentially useful fiber reinforcements may include biocompatible fibers such as glass or aramid fibers.

It should be noted that the use of polymers has distinct advantages over metal. Metal, per se, is solid and inflexible. Bone, on the other hand, is flexible and has an inherent elasticity and flexibility as its organic matrix. Bone minerals provide mechanical rigidity and load-bearing strength to the bone. However, the rigidity and inflexibility of a metal implant can, at times, be in conflict with the flexibility and elasticity of the organic matrix of the bone. The replacement implants according to the present disclosure accommodate both of these conflicting properties. As discussed herein, implants according to the present disclosure provide a combination of structural strength, stability, and durability with a low friction bearing surface for smooth movement. Manufacturing methods disclosed herein permit efficient and effective construction of replacement implants that provide these benefits. Still other benefits and advantages are recognizable to those skilled in the art.

Various embodiments of replacement implants and manufacturing methods for the same have been described herein, which include various components and features. In other embodiments, the replacement implants and manufacturing methods may be provided with any combination of such components and features. It is also understood that in other embodiments, the various devices, components, and features of the replacement implants and manufacturing methods described herein may be constructed with similar structural and functional elements having different configurations, including different ornamental appearances.

Several alternative embodiments and examples have been described and illustrated herein. A person of ordinary skill in the art would appreciate the features of the individual embodiments, and the possible combinations and variations of the components. A person of ordinary skill in the art would further appreciate that any of the embodiments could be provided in any combination with the other embodiments disclosed herein. It is understood that the invention may be embodied in other specific forms without departing from the spirit or central characteristics thereof. The present examples and embodiments, therefore, are to be considered in all respects as illustrative and not restrictive, and the invention is not to be limited to the details given herein. Nothing in this specification should be construed as requiring a specific three dimensional orientation of structures in order to fall within the scope of this invention, unless explicitly specified by the claims. When used in description of a method or process, the term “providing” (or variations thereof) as used herein means generally making an article available for further actions, and does not imply that the entity “providing” the article manufactured, assembled, or otherwise produced the article. Accordingly, while the specific embodiments have been illustrated and described, numerous modifications come to mind without significantly departing from the spirit of the invention and the scope of protection is only limited by the scope of the accompanying claims.

What is claimed is:

1. A femoral knee replacement implant, comprising:
a curvilinear base plate having at least one curvilinear posterior condyle extending upwardly therefrom and a medial portion for encircling at least a portion of a femur and for underlying a patella, the implant having an outer surface formed from a flexible polymer and an infill portion formed from a fiber-reinforced polymer, wherein the flexible polymer has greater flexibility and lower friction properties relative to the fiber-reinforced polymer, the implant being additive manufactured.

2. The implant of claim 1, wherein the implant comprises a pair of spaced apart femoral condyles having a space therebetween to accommodate ligaments passing therethrough.

3. The implant of claim 2 which further comprises at least one fiber-reinforced polymer upstanding post configured for connection to the femur.

4. The implant of claim 3, wherein the implant is a full knee replacement implant.

5. The implant of claim 1, wherein the implant is a partial knee replacement appliance having a single posterior condyle.

6. The implant of claim 1, wherein the implant is a partial knee replacement implant having a single anterior condyle.

7. The implant of claim 1, wherein the flexible polymer is selected from the group consisting of PEEK, polycaprolactone, polylactic acid and polyglycolic acid, and the fiber-reinforced polymer comprises a same material as the flexible polymer having carbon fiber reinforcement.

8. A method for additive manufacturing a femoral component implant, the method comprising:
(a) creating an outer wall of a flexible polymer from a series of at least three layers of beads of a suitable material, the layers having alternating widths to create an interstitial gap between a first lowermost layer, a second layer above the first lowermost layer and a third layer above the second layer, the first layer and third layer each having a width greater than a width of the second layer to create a gap at termini of the first and third layers, such that the first and third layers are greater width layers, and the second layer is a lesser width layer;
(b) depositing a layer of strengthening infill into the gap between the greater width layers; and
(c) repeating steps (a) and (b) sequentially to create gaps between the alternating greater width layers at the termini; and thereafter depositing a layer of strengthening infill in the respective repeated gaps;
wherein the strengthening infill extends into the gaps and into abutment with ends of the lesser width bead layers, and further wherein the greater width layers have equal numbers of beads, and the lesser width layers have equal numbers of beads, each bead in each layer being an oval bead and further wherein a bead height of the beads in a lowermost layer alternate between a first height H1 and a second height H.

9. The method of claim 8, wherein each bead in each layer above the lowermost layer is of equal height.

10. The method of claim 8, wherein each bead in each layer above the lowermost layer is of equal height and width.

11. The method of claim 8, wherein the flexible polymer is PEEK and the infill is CFR-PEEK.

12. A skeletal replacement implant configured for connection to a bone, the implant comprising:
a structural body formed of a first polymeric material, the structural body being configured to be fixedly connected to the bone to mount the implant on the bone; and
an outer layer of a second polymeric material disposed on at least a portion of the structural body to form at least a portion of an outer surface of the implant,
wherein the at least a portion of the outer surface of the implant formed by the outer layer includes a bearing surface configured to moveably engage an adjacent skeletal structure, and
wherein the first polymeric material has greater strength compared to the second polymeric material, and wherein the second polymeric material provides a lower friction surface compared to the first polymeric material.

13. The implant of claim 12, wherein the first polymer material and the second polymer material share a matrix material, and the first polymer material further includes fiber reinforcement.

14. The implant of claim 13, wherein the matrix material is PEEK, such that the first polymer material comprises carbon fiber-reinforced PEEK, and the second polymer material comprises PEEK.

15. The implant of claim 13, wherein the matrix material is selected from the group consisting of PEEK, polycaprolactone, polylactic acid and polyglycolic acid.

16. The implant of claim 12, the outer layer is disposed around an entire periphery of the structural body, such that the structural body forms an inner core of the implant.

17. The implant of claim 12, wherein the outer layer is disposed on less than an entirety of the outer periphery of the structural body, such that the outer layer is disposed at least on the bearing surface and is not disposed at least a portion of the structural body configured for connection to the bone.

18. The implant of claim 12, wherein the implant is a femoral replacement implant having at least one condyle having an outwardly-curved outer surface at least partially defining the bearing surface, and wherein the outer layer is disposed on the outwardly-curved outer surface of the at least one condyle.

19. The implant of claim 18, wherein the implant has two condyles spaced by a gap, each condyle having an outwardly-curved outer surface at least partially defining the bearing surface, and the outer layer is disposed on both of the outwardly-curved outer surfaces of the two condyles.

20. The implant of claim 12, wherein the structural body and the outer layer are formed together by an additive manufacturing technique.
